(19) United States
(12) Patent Application Publication
(10) Pub. No.: US 2003/0148296 A1
(43) Pub. Date: Aug. 7, 2003

(54) Differential gene expression in cardiac hypertrophy

(76) Inventors: Harlan Roger Brown, Durham, NC (US); Traci Ann Mansfield, Guilford, CT (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/106,691

(22) Filed: Mar. 26, 2002

Related U.S. Application Data
(60) Provisional application No. 60/280,048, filed on Mar. 30, 2001.

(51) Int. Cl. C12Q 1/68; A61K 49/00

(52) U.S. Cl. 435/6; 424/9.2

(57) ABSTRACT

Gene expression in normal unexercised hearts; chronically exercised, minimimally hypertrophied hearts; and hearts with hypertrophy due to renovascular hypertension is described. Methods of screening compounds for potential hypertrophic effects on cardiac muscle are provided.

### RELATED APPLICATION

This application claims priority to U.S. provisional application 60/280,048 filed Mar. 30, 2001.

### FIELD OF THE INVENTION

The present invention relates to gene expression in normal unexercised hearts; chronically exercised, minimimally hypertrophied hearts; and hearts with hypertrophy due to renovascular hypertension.

### BACKGROUND OF THE INVENTION

Cardiac hypertrophy is the compensatory response of the myocardium to increased work. Cardiac hypertrophy causes an increase in the overall mass and size of the heart due to an increase in the size, not the number, of individual cardiac cells. (Since adult cardiac myocytes cannot divide, changes in cardiac myocyte number cannot occur in the adult heart.) Cardiac hypertrophy can occur in several ways. Physiologic hypertrophy is induced by exercise and seems to have no deleterious effect on the heart. Pathologic hypertrophy can be caused by pressure-overload (hypertension or aortic stenosis) or volume overload (mitral regurgitation), and this can lead to myocardial contractile failure (congestive heart failure and arrhythmia). Cardiac hypertrophy is a common clinical occurrence.

### SUMMARY OF THE INVENTION

According to one embodiment of the present invention there is provided a panel of genes that are differentially expressed in cardiac hypertrophic states; such genes are useful to identify and/or distinguish “good” (exercised-induced) cardiac hypertrophy from “bad” (hypertensive-induced) cardiac hypertrophy.

### BRIEF DESCRIPTION OF THE FIGURES

The present invention will be further described by way of example and with reference to the following figures:

FIG. 1 shows genes differentially expressed in both RVhtn-induced cardiac hypertrophy hearts and Exercise-induced cardiac hypertrophy hearts.

FIG. 2 shows genes differentially expressed in RVhtn-induced cardiac hypertrophy hearts and not in Exercise-induced cardiac hypertrophy hearts.

FIG. 3 shows genes differentially expressed in Exercise-induced cardiac hypertrophy hearts vs. RVhtn-induced cardiac hypertrophy hearts.

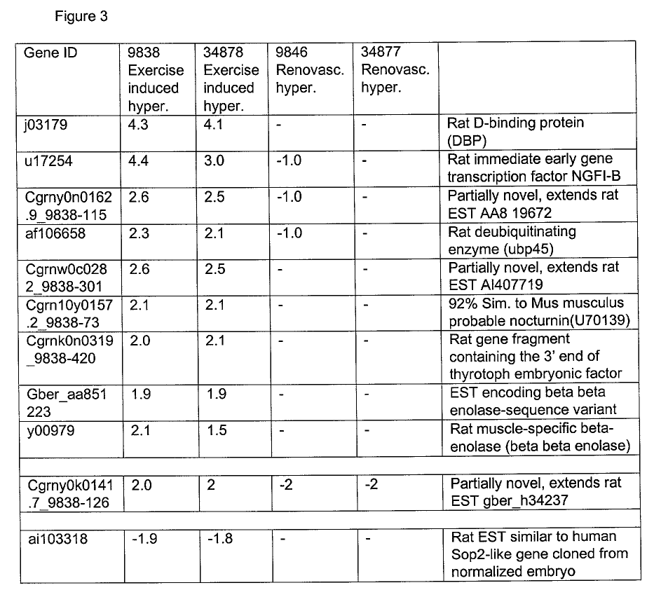

### DETAILED DESCRIPTION OF THE INVENTION

The experimental model utilized by the present inventors compared gene expression in hearts from normal unexercised animals to both animals with chronically exercised, minimally hypertrophied hearts and animals with renovascular hypertensive cardiac hypertrophy produced by the one clip/one kidney model. Delineation of gene expression patterns derived from “good” (exercised-induced) and “bad” (hypertensive-induced) cardiac hypertrophy animal models were ascertained for use as disease markers. These markers are useful in the pre-clinical evaluation and optimization of novel lead compounds.

The goal of the experiments was to determine the differential expression profile of mRNAs, using the GeneCalling technology, in hearts from normal unexercised rats to rats with chronically exercised, minimally hypertrophied hearts and rats with renovascular hypertensive cardiac hypertrophy produced by the one clip/one kidney model. Delineation of the differentially expressed genes in these two cardiac hypertrophy states identified genes that could be used as disease markers to separate “good” (exercised-induced) cardiac hypertrophy from “bad” (hypertensive-induced) cardiac hypertrophy during pre-clinical evaluation and optimization of novel lead compounds. Three sample groups were used in this study: 3 normal control rats (no cardiac hypertrophy), 3 rats with exercise cardiac hypertrophy induced through swimming in temperature-controlled water for 6 hrs/day for 6 weeks (3 hrs of swimming 2× a day with a 2 hr rest period in between), and 1 rat with renovascular hypertensive hypertrophy surgically induced using the one clip/one kidney model. Hypertrophy was assessed by heart wt/body wt ratios: Controls—0.24, 0.25, 0.25, Exercise—0.33, 0.30, 0.29 and RVhtn—0.32 (2 rats were not statistically different from controls and thus were not included in the GeneCalling study). All of the rats in the study were of the Han Wistar strain.

The three sample groups in this study were compared for analysis in 2 different ways: Exercise-induced hypertrophy vs. control (Jobs 9838 and 34878) and Renovascular hypertensive hypertrophy vs. control (Jobs 9846 and 34877). All of these job comparisons were performed at thresholds of +/−1.5-fold expression difference and a statistical significance of 85%. However, the differences in jobs 9838 and 9846 were analyzed at different times; during which time there were several improvements to the difference finding software which explains, in part, why the number of differences that passed the thresholds and the n-fold modulations are slightly different. The data from the later jobs is the most accurate.

In addition to improvements in the difference finding software, there was a sample mix-up error found in 4 of the subsequences (h0a0, i0a0, i0q0, l0h0) in the data from the original jobs 9838 and 9846. This sample mix-up problem centered on the single RVhtn sample being replaced by one of the exercise samples. This caused the misidentification of 5 genes as differentially expressed in the RVhtn sample. These genes were thrombin (m81397), C-reactive protein (m83176), 4-hydroxyphenolpyruvate (af082834), the rat ortholog of human very long-chain acyl-CoA synthetase homolog 2 (pg_rn_gbh af064255) and atrial natriuretic factor (m15868). There are two reasons that the problem was not identified earlier in the study: 1) There was only a single RVhtn sample and therefore no other samples for comparison, and 2) the exercise sample that was swapped in had its own set of differences that were not present in the other 2 exercise hypertrophy samples. The sample mix-up problem was corrected in all of the jobs, and four of the incorrectly identified genes, (thrombin, C-reactive protein, 4-hydroxyphenolpyruvate, rat VLCACS) were removed from the list of genes up-regulated in the RVhtn rat. (Once the incorrect data for ANP was removed, it was found that ANP was actually up-regulated +1.5-fold in the RVhtn sample, corresponding to previous literature reports.) A list of the genes seen to be modulated differently in the swapped Exercise hypertrophied rat (sample 72968) is shown in Table 1. This gene list indicates that this rat may have mounted an immunological response, although there is no retrospective pathology data to support this theory.

TABLE 1
Genes Differentially Expressed in Exercise Sample 72968

| Confirmed Genes | | |
|---|---|---|
| m83176 | C-reactive Protein | +9.5 |
| m81397 | Thrombin | +5.8 |
| af082834 | 4-hydroxyphenolpyruvate | +5.4 |
| m15868 | Atrial natriuretic factor (ANP) | −7.6 |
| Not Confirmed Genes (with good GeneCalls) | | |
| k01933 | Haptoglobin | +50.3 |
| m29866 | Complement component C3 | +13.6 |
| d17370 | Cystathionine gamma-lyase | +7.6 |
| x96721 | Pregnenolone 16-alpha- | +4.4 |
| | carbonitrile-inducible cypP450 | |
| x02299 | T-kininogen | +3.8 |
| k00136 | Glutathione S-transferase | +2.7 |

Over 35,200 gene fragments were assayed for expression level from an average of 85 subsequences in each job comparison. Overall, the exercise-induced hypertrophy rats showed fewer differences when compared to normal rats than the RVhtn rats did. The exercise-induced hypertrophy vs. control job comparison identified 103(0.3%)/82(0.2%) differences while the Renovascular hypertensive hypertrophy vs. control Job comparison identified 803(2.3%)/705(2.1%) differences. The greater than 7-fold increase in the number of differences in the RVhtn job vs. the Exercise job can, in part, be attributed to the smaller number of samples used in the RVhtn job (using a single RVhtn rat heart instead of triplicate samples allows for more background noise in the job comparison that would have been dampened by using triplicate samples). The larger number of differences can also be explained by the speculation that since RVhtn induced hypertrophy leads to a disease phenotype (progression toward heart failure) while exercise induced hypertrophy does not, the gene expression changes in the RVhtn cardiac hypertrophy are likely to be larger and greater in number.

Of the total number of gene expression changes identified in all of the jobs, 160 were submitted to confirmation by GeneCall/Poisoning and 27 by Isolation/Poisoning, as described herein. A total of 46 GeneCall/Poisonings (29%) were completed with a successful poisoning, and a total of 23 differentially expressed bands submitted to Isolation/Poisoning (85%) were successfully isolated and sequenced. These confirmed bands identified fragments from 26 genes known in rat, 3 genes that could be defined by their similarity to a gene with known function in another species, 3 rat ESTs, and 2 novel sequences.

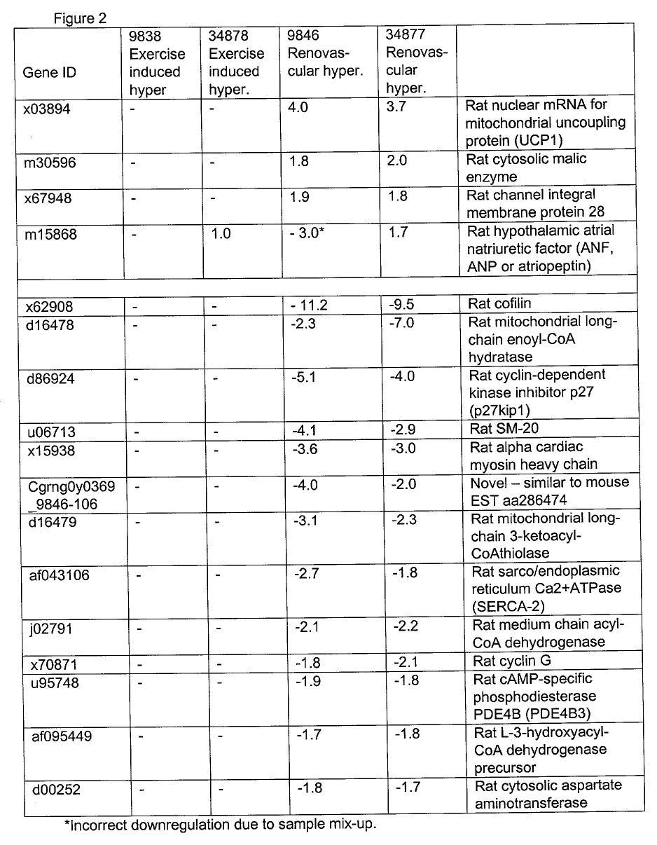

A list of genes surmised, based on the literature, to possibly have an association with cardiac hypertrophy was prepared. Of these genes, 5 were identified to be differentially expressed in this study (ANP, alpha cardiac myosin heavy chain, SERCA-2, skeletal muscle actin and cyclin G). In addition, after the differentially expressed genes in these studies were determined, another 6 of the genes found to be differentially expressed were also found to have been previously reported to be associated with hypertrophy in the literature (cyclin D2, p27kip1 and the mitochondrial beta oxidation enzymes: HAD, ECH1, ECHB and MCAD). Of these 11 genes previously reported to be associated with hypertrophy, only cyclin D2 was found by the present study to be modulated in the opposite direction from the direction reported in the literature. Also, of the 11 genes previously reported, all of them were found to be differentially expressed in the renovascular hypertension-induced cardiac hypertrophy (RVhtn) samples and not changed in the exercise-induced cardiac hypertrophy samples (See FIG. 2). This may be a reflection of the lack of studies that have been reported describing gene expression changes in cardiac hypertrophy caused by exercise.

Differentially Expressed Genes

The goal of this study was to determine whether physiologic cardiac hypertrophy and pathologic hypertrophy could be differentiated by gene expression patterns and if so, could these genes be identified for use as a panel of markers to differentiate these two hypertrophy states. We have shown that physiologic and pathologic hypertrophy can be delineated at the level of gene expression patterns. Firstly, there are far fewer genes changing in the physiologic hypertrophy model compared to the pathologic hypertrophy model (0.2% of assayed bands changed in the exercise samples vs. 2.0% in the RVhtn samples). In the exercise job comparison (34878), there were 82 differences +/−1.5-fold or greater and 24 of these differences were +/−2.0-fold or greater. In the RVhtn job comparison (34877), there were 705 differences +/−1.5-fold or greater and 263 of these differences were +/−2.0-fold or greater.

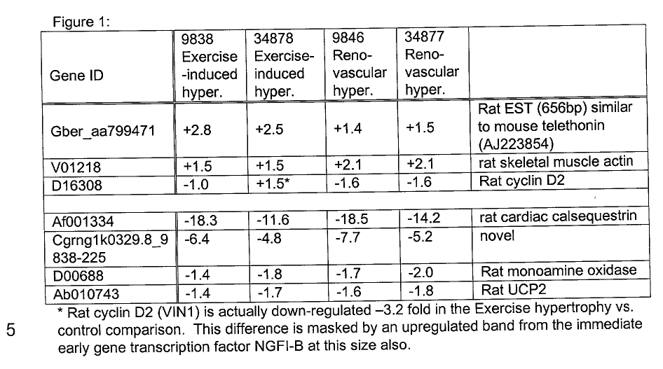

Secondly, of the 34 genes identified to be differentially expressed in this study, we only confirmed 7 that were differentially expressed in the same direction in both of the cardiac hypertrophy models (See FIG. 1; up-regulated—telethonin and skeletal muscle actin; down-regulated—cyclin D2, calsequestrin isoform, novel gene fragment, MOA and UCP2). There were an additional 7 bands down-regulated in both exercise and RVhtn and 6 bands up-regulated. However, since this pattern of expression was not of interest in determining gene expression patterns that could be used to differentiate physiological and pathological hypertrophy, these differences were not pursued further.

A total of 17 genes were confirmed that were differentially expressed only in the RVhtn samples, 9 genes were confirmed that are only differentially expressed in the exercise samples and 1 gene was identified that was modulated in opposite directions in the RVhtn samples vs. the exercise samples. From this group, a panel of 20 cardiac hypertrophy marker genes has been compiled (See Table 2). These genes can be examined in experiments where cardiac hypertrophy is induced by lead compounds or drug candidates and they will be predictive in determining whether the hypertrophy induced by the compound aligns with the physiologic cardiac hypertrophy model (induced by exercise) or the pathologic cardiac hypertrophy (induced by renovascular hypertension).

TABLE 2
20 Cardiac Hypertrophy Marker Genes

| “Good” Hypertrophy Markers | | |
|---|---|---|
| u17254 | NGFI-B | +4 |
| j03179 | D-binding protein | +4 |
| s58745 | TEF | +2 |
| y00979 | Beta beta enolase | +2 |
| isolated | Rat nocturnin | +2 |
| af106658 | UBP45 | +2 |
| aa819672 | rat EST (458 bp) | +2 |
| ai407719 | rat EST (640 bp) | +2 |
| ai103318 | rat EST sim. to Sop2-like gene | −2 |
| “Bad” Hypertrophy Markers | | |
| x03894 | UCP1 | +4 |
| m30596 | Cytosolic malic enzyme | +2 |
| m15868 | ANP | +1.5 |
| X62908 | Cofilin | −10 |
| d86924 | p27kipl | −4 |
| u06713 | SM-20 | −3 |
| x15938 | MYH6 | −3 |
| d16479 | Mitochondrial long-chain | −3 |
| | 3-ketoacyl-CoA thiolase | |
| j02791 | Medium chain acyl-CoA | −2 |
| | dehydrogenase | |
| af043106 | SERCA-2 | −2 |
| x70871 | Cyclin G | −2 |

Gene expression changes caused by pathological cardiac hypertrophy has been extensively studied. It is known that pathologic hypertrophy induces large changes in energy metabolism in cardiomyocytes and modified contractile properties in the myocardium. There is a decrease in the rate of calcium uptake into the sarcoplasmic reticulum and an alteration in the speed of relaxation of the hypertrophied heart muscle. An increase in the ratio of beta myosin heavy chain isoform to the alpha myosin heavy chain isoform results in a slower rate of ATP cycling and ultimately a slower velocity of contraction and relaxation and an improved economy of cardiac pumping function. Most of the 17 genes identified that are only differentially expressed in the RVhtn sample and not changed in the exercise samples fit into this model.

Genes Specific to Exercise-induced Hypertrophy

Nine genes were found to be differentially expressed specifically in response to Exercise-induced hypertrophy and not changed in response to Renovascular hypertension-induced hypertrophy. Expression of eight of the genes was increased while expression of one of the genes was decreased. In addition, there was one gene that was modulated in opposite directions in the exercise-induced hypertrophy rats and the RVhtn-induced hypertrophy rats. Together, these ten genes give a profile of expressed genes specific to exercise-induced hypertrophy that can be used as markers to distinguish exercise-induced or “good” hypertrophy from renovascular hypertension-induced or “bad” hypertrophy”. Some of the more interesting of these genes are discussed below.

Rat Immediate Early Gene Transcription Factor NGFI-B (GenBank u17254)

NGFI-B (nerve growth factor-induced-B, also called Nur77 or TIS-1) is an immediate early gene originally identified due to rapid, transient induction in the rat pheochromocytoma cell line PC12 by nerve growth factor (NGF) (Milbrandt, 1988). NGFI-B has structural features of a ligand-activated transcriptional regulator and is a member of the NGFI-B subfamily of nuclear receptors. Other NGFI-B subfamily members are Nur-related factor 1 (Nurr1) and Neuron derived orphan receptor (NOR-1).

NGFI-B (1692 bp) encodes a 564aa, 61 kD protein encoding an orphan nuclear receptor that is constitutively expressed in adult rat tissues with highest levels of mRNA found in the pituitary and high levels found in the cerebral cortex, muscle, ventral prostate, thymus and adrenal glands (Bandoh et al., 1997). NGFI-B mRNA is expressed in heart at low but detectable levels (approx. 100 attomoles/mg total RNA). NGFI-B expression can be induced by a variety of stimuli including stressors, cAMP, phorbol ester, growth factors, peptide hormones, neurotransmitters as well as physical stimulation such as membrane depolarization, mechanical agitation and a magnetic field (Maruyama et al., 1998). NGFI-B expression in the brain has also been shown to be induced in vivo by various treatments and insults [for example, NGFI-B is induced in the cerebral cortex, midbrain, and cerebellum of animals that experienced a convulsant-induced seizure (Watson and Milbrandt, 1989)]. NGFI-B has also been reported to be induced by light in the suprachiasmatic nucleus of the hypothalamus (Rusak et al., 1992; Morris et al., 1998), expressed at high levels and involved in induction of apoptosis in T-cells and T-cell hybrids (Woronicz et al, 1994; Liu et al., 1994), induced during the early stage of glomerulonephritis (Hayashi et al., 1996) and in small-cell lung cancer tumors (Ueda et al., 1999). However, NGFI-B knockout mice thrive and reproduce normally (Crawford et al., 1995). NGFI-B has been reported to regulate steroid hydroxylase transcription (no changes seen in this study; rat 21-hydroxylase mRNA, GenBank u56853, GeneCall: 0 of 9), corticotropin releasing hormone (CRH) gene (no changes seen in this study; rat corticotropin releasing hormone, GenBank m54987, GeneCall: 0 of 10) and human brain fructose-biphosphate aldolase C (no changes seen in this study; rat brain mRNA for aldolase C, GenBank x06984, GeneCall: 0 of 1). Previous studies have shown that NGFI-B expression can be induced in skeletal muscle cell lines (Lim et al., 1995) but is not induced in mouse gastrocnemius muscle by electrical stimulation of the sciatic nerve in a pattern of brisk intermittent exercise (Abu-Shakra, 1993).

NGFI-B response element (NBRE; 5′-AAAGGTCA-3′ or 5′-GAATGCCA-3′) NGFI-B can bind to the NBRE as a monomer or heterodimerize with RXR and bind retinoic acid response elements to activate transcription (no changes seen in this study; rat retinoid X receptor alpha, GenBank I06482, GeneCall: 0 of 8). Also, homodimers of NGFI-B have been reported to bind to a novel response element (NurRE), within the pro-opiomelanocortin gene (POMC) promoter (no changes seen in this study; rat pro-opiomelanocortin gene, GenBank k01877, GeneCall: 0 of 6). Both a truncated form of NGFI-B lacking the putative ligand binding domain and full-length NGFI-B could activate transcription from a reporter plasmid in experiments in COS cells (Wilson et al., 1991). It was postulated that the NGFI-B ligand may be synthesized by COS cells, that a ligand may not be required by NGFI-B in order to activate transcription or the activation may have been weak compared to activation with a ligand.

Identification of NGFI-B as the gene with the highest-fold induction in the exercise hypertrophied rats is a very interesting finding and has not been previously reported. Induction was only seen after 6 weeks of exercise training and subsequent cardiac hypertrophy and not in the RVhtn sample. (Transcriptional activation has previously been reported to have an immediate-early component, which can occur in the absence of de novo protein synthesis, and a delayed-early component, which is dependent on de novo protein synthesis—Williams and Lau, 1993).

There was no full-length mRNA sequence available for rat NGF, only genomic sequence containing the second (coding) exon. This exon was not detected using any of the CuraGen subsequences. It would be of interest to assay the exercise hypertrophy-induced cardiac tissue to determine whether there is increased levels of NGF protein that is contributing to the upregulation of NGFI-B.

Rat D-binding Protein (DBP) (GenBank j03179)

DBP is a member of the PAR subfamily of basic/leucine zipper (bZip) transcription factors (the two other family members are Thyrotroph Embryonic Factor, TEF—also identified as up-regulated approximately 2-fold in this study, and Hepatic Leukemia Factor, HLF). The PAR domain is a conserved proline- and acidic-rich domain to the immediate amino-terminal side of the basic domain. Significant protein expression of DBP is confined to the liver, however DBP mRNA is present in most tissues (excluding testis). DBP was cloned due to its ability to bind to the D-site in the albumin promoter and activate transcription in the adult liver (Mueller et al., 1990). Expression of DBP is down-regulated upon induction of regenerative growth and dedifferentiation. DBP has also been reported to bind the promoters and transactivate several other genes expressed in the liver including cholesterol 7 alpha hydroxylase, cytochrome P450 CYP2C6, alcohol dehydrogenase, serpin, aldolase B and phosphoenolpyruvate carboxykinase (PEPCK) (Roesler et al., 1992). The ability of DBP to bind and activate the PEPCK promoter is especially interesting since PEPCK catalyzes the formation of phosphoenolpyruvate from oxaloacetate in the gluconeogenesis pathway showing that DBP can transcriptionally regulate an enzyme in a metabolic pathway. Transcription of phosphoenolpyruvate carboxykinase (PEPCK) has also been reported to be stimulated by retinoic acid and RAR, cAMP or thyroid hormone tri-iodothyronine with CCAAT-enhancer-binding protein (C/EBP) (Park et al., 1997) showing cooperativity of a transcription factor and nuclear receptors at affecting transcription of an enzyme. However, the ability of DBP to affect transcription of PEPCK has been used to explain the highest expression levels of PEPCK in the liver (contradicts our finding of induction of expression of DBP in the exercise-hypertrophied heart). Expression of liver-enriched transcription factors, including DBP, has been seen in cardiac mesoderm during fetal development (Van den Hoff et al., 1994) indicating that increased DBP expression during exercise-induced cardiac hypertrophy may be due to a reversion to a fetal expression pattern. DBP has been shown to have circadian expression patterns (Fonjallaz et al., 1996). Several of the genes identified to be modulated by exercise-induced cardiac hypertrophy have been shown to play a role in circadian rhythm (nocturnin, DBP and TEF). This may indicate a disruption of normal circadian rhythm gene expression in the exercise-induced hypertrophied heart.

Rat Muscle-specific Beta-enolase (Beta Beta Enolase) (GenBank y00979 & aa851223)

There are 3 isozymes of enolase (2-phospho-D-glycerate hydrolase) that occur in mature mammalian tissues: alpha alpha—called non-neuronal enolase; gamma gamma—neuron-specific enolase; and beta beta—muscle-specific enolase. There has been a muscle-specific enhancer identified within the first intron of the human beta enolase gene (Feo et al., 1995). Enolase is an enzyme involved in the last stage of the glycolytic pathway that catalyzes the dehydration of 2-phosphoglycerate to phosphoenolpyruvate (one step prior to the formation of pyruvate and generation of the second ATP molecule). AA851223 is a 521 bp EST with 98% identity to beta beta enolase (y00979). It contains a point mutation difference from y00979 that creates a RE site and fragment m1n0-181.2, not seen in y00979. Beta beta enolase is most highly expressed in adult skeletal muscle. In a simplistic view, a switch from alpha alpha (non-neuronal) enolase to beta beta (muscle-specific) enolase expression occurs during the final stage of cell differentiation and beta beta enolase expression increases with the functional maturation of myotubes. However, the hybrid alpha beta enolase still contributes approximately 30% to total enolase activity in the adult heart. It has been reported that during pathological cardiac growth following aortic stenosis, there is a downregulation of the beta enolase gene leading to a less mature cardiac phenotype-increased alpha to beta enolase subunit ratio (Keller et al., 1995). This is exactly the opposite effect that was seen in the exercise-induced hypertrophied hearts. No change in expression of any of the enolase isozyme genes were seen in the RVhtn-hypertrophied hearts. It has been reported that enolase activity is low in adult rat liver and only the alpha alpha enolase isozyme is expressed (Keller et al., 1995).

The set of 20 Cardiac Hypertrophy Marker Genes (Table 2), or a subset thereof, will be useful to evaluate drug compounds that cause hypertrophy to determine whether the hypertrophy resembles exercise-induced hypertrophy and would therefore be considered “Good” or if it resembles Renovascular-hypertension-hypertrophy and would therefore be considered “Bad”. These 20 genes would most likely be used in a screening panel to examine their expression in heart tissue from rats or other mammals treated with the compounds under investigation.

The set of 9 genes that were differentially expressed in the exercise-hypertrophy samples (see Table 2), or a subset thereof, are useful to determine whether cardiac hypertrophy is similar to exercise-induced cardia hypertrophy. Although there have been many studies outlining the changes in gene expression in the heart due to volume or pressure-overload-induced hypertrophy, there are few studies that address the gene changes in exercise-induced cardiac hypertrophy, and none of the genes in the present study have been previously associated with “Good” hypertrophy. In particular, NGFI-B is an orphan nuclear receptor with activity that could be modulated by a small molecule compound. There are two novel sequences in this group, cgrny0n0162.9_{—}9838-115 and cgrnw0c0282_{—}9838-301, that extend rat ESTs but at this point only represent gene fragments and not full-length genes.

### EXPERIMENTAL

### Example 1

### Materials and Methods

Organism Source: rattus norvegicus

Tissue Source: heart

Sample Groups Submitted:

| Sample Group | | ID#'s | Strain | Organ | Treatment |
|---|---|---|---|---|---|
| 1. | Control | 72965/72966/ | Han Wistar | Heart | Control |
| | | 72967 | rats | | |
| 2. | Exercise | 72968/72969/ | Hans Wistar | Heart | Exercise |
| | | 72970 | rats | | |
| 3. | RVhtn | 72971 | Hans Wistar | Heart | Renovascular |
| | | | rats | | hypertrophy |

RNA was isolated from the samples by grinding frozen tissue in liquid Nitrogen, followed by extraction of 2 grams of powder and purification of the total RNA using a standard Trizol protocol. Yields of total RNA for each sample passed the usual QC evaluations. The OD260/280 ratio was in the range of 1.6-1.8 and denaturing gel electrophoresis indicated that the ratio of the cytoplasmic ribosomal RNAs was correct. mRNA was purified from 50 micrograms of total RNA using oligo-dT magnetic beads, with yields ranging from 1832 to 2878 nanograms.

Genomics Facility Processing Specifications:

| Sample Group | Sample #s | ng cDNA | # Subsequences |
|---|---|---|---|
| Control | 72965/72966/72967 | 982/951/1134 | 87 |
| Exercise | 72968/72969/72970 | 950/1107/953 | 86 |
| RVhtn | 72971 | 777 | 85 |

GeneCalling chemistry was performed on 1 ng of cDNA per subsequence pair. The GeneCalling method has 3 main steps: restriction endonuclease digestion, adapter ligation and PCR amplification. Briefly, the cDNA was digested with a standard set of 96 different pairs of restriction enzymes with 6 base-pair recognition sites (subsequence pairs). Complementary adapters were ligated to the digested cDNA and adapter-specific primers were used to direct 20 cycles of PCR, amplifying fragments containing sites for the pairs of restriction enzymes used. One adapter-specific primer is biotin-labeled while the other is labeled with the fluorescent dye FAM. Following PCR amplification, the biotin labeled DNA was purified on immobilized streptavidin. Denatured single-stranded DNA fragments were resolved by capillary electrophoresis on MegaBace instruments, and FAM-labeled fragments were detected upon laser excitation. Since the biotin label is necessary for purification and the FAM label is necessary for detection, all detected fragments result from restriction digestion with both enzymes. The output of the electrophoresis instruments was processed using the Java-based internet-ready Open Genome Initiative (OGI) software suite. Three independent reactions from the same cDNA sample were compared for quality of electrophoretic peak resolution and reproducibility of peak patterns. Composite traces from each sample were generated and then compared between three independent samples for peak quality and reproducibility. The resulting traces, placed in an Oracle database, represent the total gene expression profile for the tissue sampled and treatment in question. The databases for each sample can be compared to identify differences in gene expression resulting from the different cardiac hypertrophy treatments. The composite traces calculated for each sample group, based on average peak height and variance, were compared among sample groups using software designed to identify peaks representing differential expression.

### Example 2

### Analysis Strategy

Goal: To identify genes that can be used as markers for “good” hypertrophy (i.e. exercise-induced, in this study) vs. “bad” hypertrophy (renovascular hypertension -induced).

Jobs:

Exercise-induced hypertrophy vs. control, # diffs=103 (0.3%)

Exercise-induced hypertrophy vs. control, # diffs=82 (0.2%)

Renovascular hypertensive hypertrophy vs. control, diffs=803 (2.3%)

Renovascular hypertensive hypertrophy vs. control, # diffs=705 (2.1%)

Analysis Strategy:

a. Focused on known genes that were increased or decreased in expression in exercise vs. control and not changed or modulated in the other direction in the renovascular hypertension vs. control job.

b. Focused on known genes that were increased or decreased in expression in renovascular hypertension vs. control and not changed or modulated in the other direction in the exercise vs. control job.

In the four job comparisons, at least 34,000 bands per comparison were assayed. Jobs were run at stringency settings of N-fold difference cut-off greater than 1.5-fold in either direction and band significance of 0.85 or greater. All differences were visually inspected to ensure that the difference-finding algorithm had not identified questionable differences, shoulder peaks or occasional noise. The average number of subsequences used in the comparison was 85 (out of a total of 96).

Confirmation and gene identification of differentially expressed bands can occur by two routes. The most efficient method is GeneCalling/Poisoning. In this case, cDNA fragments representing differentially expressed genes can be identified by database searching with the 6 base-pair restriction enzyme recognition sequences at the fragment ends and the exact length of each fragment (determined electrophoretically, subtracting linker length). Database searching for genes predicted to have restriction fragments of matching lengths enables the immediate identification of all of the genes whose sequences reside in that database and “flags” fragments derived from novel genes by virtue of their absence from the database. Given a three nucleotide size window, database lookup can provide a unique assignment of gene identity. This single hit is often referred to as a “GeneCall”. The detection of multiple fragments derived from the same gene which show differential expression of the same directional modulation increases the likelihood that the prediction of the gene identity is correct.

The differentially expressed gene fragment and the gene, cDNA or EST sequence identified by the database lookup, are unequivocally linked through a positive poisoning reaction. In this process, the reaction containing the fragment of interest is performed a second time using the same end primers, but in the presence or absence of an excess of an unlabeled oligonucleotide whose sequence is derived from the predicted gene fragment. If the identity of the fragment was predicted correctly, the unlabeled oligo will out-compete the universal oligo for priming that fragment and appear in the chromatogram to ablate that peak specifically without affecting the amplification of the other fragments. The efficiency and success of the GeneCalling/Poisoning method relies predominantly on the quality of the database used. A database with large contigs assembled from EST sequences or a high proportion of full length sequences increase the probability that a gene is correctly identified and confirmed using the poisoning reaction. In this study, differentially expressed gene fragments were GeneCalled against the CuraGen SeqCalling Rat Assemblies (SCDB3) database, as well as the GenBank Rat and GenBank Rat Patent databases.

An alternative but more time consuming method for gene identification and confirmation, Isolation/Poisoning, relies on the isolation of the differentially expressed fragment from the re-amplified GeneCalling chemistry reaction from a preparative gel. The gel-purified fragment is re-amplified and cloned in a standard PCR product cloning vector. The insert is sized and fragment. The Poisoning reaction is performed and analyzed as described above. Successful ablation of the peak using the unlabeled oligonucleotide based on the cloned sequence unambiguously identifies the sequence as corresponding to the original differentially expressed gene fragment. Subsequently, the gene identification is obtained by standard BLASTN or BLASTX analysis of the poisoning cloned sequence, against the CuraGen SeqCalling Rat Assemblies (SCDB3) database, as well as GenBank non-redundant DNA and protein databases. The BLAST analysis, and additional sequence analysis discussed herein, is conveniently performed within the GeneScape environment. Hits to genes from a different species are recognized with a “Similar to” (Sim.) identity assignment. The assignment of a “NOVEL” identification to a gene fragment is the result of either the complete absence of any BLAST hits or a BLASTN result with p>1e-5.

### Example 3

### Results

10.1 Job Array 34889: Exercise vs. RVhtn

Jobs

Job 9838: Exercise-induced hypertrophy vs. control

Job 34878: Exercise-induced hypertrophy vs. control

Job 9846: Renovascular hypertensive hypertrophy vs. control

Job 34877: Renovascular hypertensive hypertrophy vs. control

Band Statistics for Array 34889

| | | | N-fold | | Total | Differentially | Locked | Bands | Genes |
|---|---|---|---|---|---|---|---|---|---|
| Job | Set A | Set B | Thresholds | Subsequences | Bands | Expressed Bands | Bands | Confirmed | Confirmed |
| 9838 | Rat hyper | rat hyper | +1.5/−1.5 | 86 | 35611 | 103 | 9 | 27 | 19 |
| | exer | ctrl | | | | | | | |
| 34878 | Rat hyper | rat hyper | +1.5/−1.5 | 86 | 35599 | 82 | 0 | 0 | 0 |
| | exer | ctrl | | | | | | | |
| 9846 | Rat hyper | rat hyper | +1.5/−1.5 | 85 | 35201 | 804 | 38 | 39 | 26 |
| | reno | ctrl | | | | | | | |
| 34877 | Rat hyper | rat hyper | +1.5/−1.5 | 83 | 34405 | 705 | 0 | 1 | 1 |
| | reno | ctrl | | | | | | | |

Confirmation Status for Array 34889

| | | Isolations | Isolations | Isolations | Isolations | Poisons | Poisons | Poisons | Poisons | Poisons |
|---|---|---|---|---|---|---|---|---|---|---|
| Job | Job Title | Requested | Completed | In Process | Dropped | Requested | Passed | Failed | Dropped | In Process |
| 9838 | Exercise- | 16 | 14 | 0 | 2 | 39 | 13 | 22 | 4 | 0 |
| | induced | | | | | | | | | |
| | hypertrophy | | | | | | | | | |
| | vs. control | | | | | | | | | |
| 34878 | Exercise- | 0 | 0 | 0 | 0 | 0 | 0 | 0 | 0 | 0 |
| | induced | | | | | | | | | |
| | hypertrophy | | | | | | | | | |
| | vs. control | | | | | | | | | |
| 9846 | Renovascular | 11 | 9 | 0 | 2 | 118 | 30 | 75 | 13 | 0 |
| | hyper-tensive | | | | | | | | | |
| | hypertrophy | | | | | | | | | |
| | vs. control | | | | | | | | | |
| 34877 | Renovascular | 0 | 0 | 0 | 0 | 3 | 1 | 0 | 0 | 2 |
| | hyper-tensive | | | | | | | | | |
| | hypertrophy | | | | | | | | | |
| | vs. control | | | | | | | | | |

Gene List for Array 34889

| | | | | | | | |
|---|---|---|---|---|---|---|---|
| Gene Name | Accno | Bands | 9838 | 34878 | 9846 | 34877 | Description |

01.05.02 Ubiquination

| | | | | | | | |
|---|---|---|---|---|---|---|---|
| UBP45: Rat | af106658 | i0c0- | +2.3 | +2.1 | — | — | Rat UBP45 is a deubiquiti- |
| deubiquitinating | | 220.1 | | | | | nating enzyme that was orig- |
| Enzyme (ubp45) | | i010- | | | | | inally cloned from skeletal |
| | | 166.5 | | | | | muscle. Deubiquitinating |
| | | m0r0- | | | | | enzymes are cysteine pro- |
| | | 61.4 | | | | | teases that cleave ubiquitin |
| | | | | | | | from ubiquitin-conjugated |
| | | | | | | | protein substrates. There are |
| | | | | | | | more than 90 identified |
| | | | | | | | deubiquitinating enzymes with |
| | | | | | | | significant sequence diver- |
| | | | | | | | sity indicating a broad range |
| | | | | | | | of substrate specificities. |
| | | | | | | | Medline 10603300, |
| | | | | | | | TrEMBL AAF14189, |

Job 9838
Comment 1: Gene fragments are actually identical to both rat UBP45 (af106658 & af202454) and rat UBP69
(af106659 & af202453). UBP45 and UBP69 are identical over the 3′ end of their sequence (about 1080 bp
out of 2095 bp total for UBP69). Both were cloned from skeletal muscle. There is also a very high
identity over the 3′ half of the sequence to mouse, human and chicken UBP41.
02.01.01 Peptide Hormones

| | | | | | | | |
|---|---|---|---|---|---|---|---|
| ANF:Rat | m15868 | i0a0- | — | 1 | −3 | +1.7 | ANF is a potent vasoactive |
| hypothalamic | | 135.5 | | | | | peptide synthesized and |
| atrial natriuretic | | | | | | | secreted by mammalian heart |
| factor (ANF, ANP | | | | | | | atria in response to volume |
| or atriopeptin) [C] | | | | | | | expansion. ANF inhibits |
| | | | | | | | sodium reabsorption in the |
| | | | | | | | distal tubules of the kidney |
| | | | | | | | and causes vasodilation |
| | | | | | | | playing a key role in |
| | | | | | | | cardiovascular homeo- |
| | | | | | | | stasis. ANF has a CGMP- |
| | | | | | | | stimulating activity. |
| | | | | | | | Medline 2951736, |
| | | | | | | | Swiss-Prot P01161. |

Job 9846
Comment 1: Original down regulation of ANP in this sample has been attributed to a sample mix-up. The
differences were failed.
Comment 2: In most reported studies, increased ventricular mass has been associated with high
ventricular expression of ANF. PMID 10381898
02.12.04 Cyclic Nucleotide Phosphodiesterases

| | | | | | | | |
|---|---|---|---|---|---|---|---|
| PDE4B3: Rat | u95748 | g1k0- | — | — | −1.9 | −1.8 | Cyclic AMP, a second mes- |
| cAMP-specific | | 370.4 | | | | | senger in the action of many |
| phosphodiesterase | | | | | | | hormones, is formed from ATP |
| PDE4B | | | | | | | by adenylate cyclase and de- |
| (PDE4B3) | | | | | | | graded by a specific phospho- |
| | | | | | | | diesterase. At least seven |
| | | | | | | | different cyclic nucleotide |
| | | | | | | | phosphodiesterases are known |
| | | | | | | | to exist in mammalian |
| | | | | | | | tissues. The PDE4 gene |
| | | | | | | | family is one of the largest |
| | | | | | | | with isoforms expressed in |
| | | | | | | | many tissues including |
| | | | | | | | skeletal muscle, airway |
| | | | | | | | smooth muscle cells, brain, |
| | | | | | | | lymphocytes, liver and |
| | | | | | | | kidney. PDE4 is the primary |
| | | | | | | | membrane-bound phospho- |
| | | | | | | | diesterase found in rat |
| | | | | | | | cardiac muscle. PDE4B3 is a |
| | | | | | | | 721aa “long form” PDE4B |
| | | | | | | | splice variant with a |
| | | | | | | | unique 79aa N-terminal |
| | | | | | | | region. |
| | | | | | | | Medline 1848733, 7480160, |
| | | | | | | | 9371714, |

Job 9846
Comment 1: There are no literature reports associating changes in PDE4 gene expression with cardiac
hypertrophy.
02.14.01 Transcription Factors

| | | | | | | | |
|---|---|---|---|---|---|---|---|
| TEF: Rat gene | Cgrnk0n0319_9838- | k0n0- | +2 | +2.1 | — | — | Novel rat gene fragment (319 |
| fragment | 420 | 319 | | | | | bp) that overlaps the 3′-most |
| containing the 3′- | | | | | | | 12 nucleotides of rat thyro- |
| end of thyrotroph | | | | | | | troph embryonic factor (TEF; |
| embryonic factor | | | | | | | GenBank entry S58745 which |
| (S58745) [N] | | | | | | | contains the sequence for rat |
| | | | | | | | TEF CDS but no 3′UTR |
| | | | | | | | sequence). TEF is a member of |
| | | | | | | | the PAR (proline and acidic |
| | | | | | | | amino acid-rich) subfamily of |
| | | | | | | | basic leucine zipper (bZip) |
| | | | | | | | transcription factors. TEF is |
| | | | | | | | expressed exclusively in the |
| | | | | | | | anterior pituitary during |
| | | | | | | | embryogenesis but is found in |
| | | | | | | | several tissues in juvenile |
| | | | | | | | and adult rats. TEF can bind |
| | | | | | | | DNA as a homodimer or as a |
| | | | | | | | heterodimer with DBP. TEF and |
| | | | | | | | DBP have both been implicated |
| | | | | | | | to play roles in circadian |
| | | | | | | | rhythms. |
| | | | | | | | Medline 1916262, 8617210, |
| | | | | | | | Swiss-Prot P41224, |

Job 9838
Comment 1: A contig can be made spanning this gene fragment and the rat TEF CDS (S58745) by using
2 mouse ESTs (Al892971 and AA138848) that are 96% identical at the nucleotide level to rat TEF
(S58745). No other bands were predicted to be detected with the standard 96 subsequence analysis.

| | | | | | | | |
|---|---|---|---|---|---|---|---|
| DBP: Rat D- | j03179 | m0v0- | +4.3 | +4.1 | — | — | Rat D-binding protein (DBP) |
| binding protein | | 193.4 | | | | | is a member of the PAR sub- |
| (DBP) | | | | | | | family of basic leucine |
| | | | | | | | zipper (bZip) transcription |
| | | | | | | | factors. Significant expres- |
| | | | | | | | sion of DBP is confined to |
| | | | | | | | the liver, however DBP mRNA |
| | | | | | | | is present in most tissues |
| | | | | | | | (excluding testis). DBP has |
| | | | | | | | been reported to bind the |
| | | | | | | | promoters and transactivate |
| | | | | | | | several genes expressed in |
| | | | | | | | the liver including albumin, |
| | | | | | | | cholesterol 7 alpha |
| | | | | | | | hydroxylase, cytochrome P450 |
| | | | | | | | CYP2C6, phosphoenolpyruvate |
| | | | | | | | carboxykinase and angio- |
| | | | | | | | tensinogen. DBP has been |
| | | | | | | | shown to have circadian |
| | | | | | | | expression patterns. PMID: |
| | | | | | | | 1059357 Medline 91249397, |

Job 9838
Comment 1: DBP has been reported to be expressed in embryonic cardiac mesoderm (prior to hepatic
endoderm formation). Histochem J 26:20-31, 1994
02.14.02 Nuclear Hormone/Orphan Receptors

| | | | | | | | |
|---|---|---|---|---|---|---|---|
| NGFI-B: Rat | u17254 | k0n0- | +4.4 | +3 | — | — | NGFI-B (nerve growth factor- |
| immediate early | | 78.6 | | | | | induced-B) is an immediate |
| gene transcription | | l0w0- | | | | | early gene originally |
| factor NGFI-B | | 97.7 | | | | | identified due to rapid, |
| | | w0c0- | | | | | transient induction by nerve |
| | | 108.1 | | | | | growth factor (NGF). NGFI-B |
| | | | | | | | (1692 bp) encodes a 564 aa, |
| | | | | | | | 61 kd protein encoding an |
| | | | | | | | orphan nuclear receptor that |
| | | | | | | | can be induced by a variety |
| | | | | | | | of stimuli, including |
| | | | | | | | stressors. NGFI-B has been |
| | | | | | | | reported to regulate steroid |
| | | | | | | | hydroxylase transcription. |

03.03.02 Cyclins

| | | | | | | | |
|---|---|---|---|---|---|---|---|
| Cyclin D2: Rat | d16308 | l0e1- | — | +1.5 | −1.6 | −1.6 | Cyclin D2 is normally ex- |
| cyclin D2 | | 134.3 | | | | | pressed in the G1 phase of |
| | | | | | | | the cell division cycle and |
| | | | | | | | promotes progression through |
| | | | | | | | G1 of the cell cycle. D-type |
| | | | | | | | cyclins assemble with cyclin- |
| | | | | | | | dependent kinases (CDK4 and |
| | | | | | | | CDK6) to form holoenzymes |
| | | | | | | | that facilitate exit from G1 |
| | | | | | | | by phosphorylating key sub- |
| | | | | | | | strates (including Rb). |

Job 9846
Comment 1: Previous report associated an upregulation of cyclin D2 with LV hypertrophy in rats (days
3 to 21 post aortic constriction). Am J Physiol 275(3 Pt 2):H814-22, 1998

| | | | | | | | |
|---|---|---|---|---|---|---|---|
| Cyclin D2: Rat | I09752 | g0y0- | +1.7 | +1.5 | −2.7 | −2.3 | Cyclin D2 is normally ex- |
| cyclin D2 (VIN1) | | 152 | | | | | pressed in the G1 phase of |
| [C] | | l0w0- | | | | | the cell division cycle and |
| | | 97.7 | | | | | promotes progression through |
| | | | | | | | G1 of the cell cycle. D-type |
| | | | | | | | cyclins assemble with cyclin- |
| | | | | | | | dependent kinases (CDK4 and |
| | | | | | | | CDK6) to form holoenzymes |
| | | | | | | | that facilitate exit from G1 |
| | | | | | | | by phosphorylating key sub- |
| | | | | | | | strates (including Rb). |

Job 9838
Comment 1: Rat cyclin D2 (VIN1) is actually down regulated −3.2 fold in this job. This difference is
masked by an upregulated band from the immediate early gene transcription factor NGFI-B at this size
also.
Job 9846
Comment 1: Previous report associated an upregulation of cyclin D2 with LV hypertrophy in rats (days 3
to 21 post aortic constriction). Am J Physiol 275(3 Pt 2):H814-22, 1998

| | | | | | | | |
|---|---|---|---|---|---|---|---|
| Cyclin G: Rat | x70871 | l0e1- | — | — | −1.8 | −2.1 | Cyclin G was identified as a |
| cyclin G. | | 343.3 | | | | | target of the p53 tumor sup- |
| | | | | | | | pressor protein (levels of |
| | | | | | | | cyclin G are increased after |
| | | | | | | | induction of p53 by DNA |
| | | | | | | | damage.) The function of |
| | | | | | | | cyclin G has not yet been |
| | | | | | | | established but it may be |
| | | | | | | | associated with growth |
| | | | | | | | stimulation. Cyclin G has |
| | | | | | | | homology to S. pombe Cig1, a |
| | | | | | | | B-type cyclin. Overexpression |
| | | | | | | | of cyclin G may play a role |
| | | | | | | | in facilitating apoptosis. |
| | | | | | | | Medline 10467405, Swiss-Prot |
| | | | | | | | p39950, |

03.03.03 Cyclin Dependent Kinase Inhibitors

| | | | | | | | |
|---|---|---|---|---|---|---|---|
| p27kip1: Rat | d86924 | d0v0- | — | — | −5.1 | −4 | The p27kip1 protein binds to |
| cyclin-dependent | | 129.3 | | | | | and inhibits complexes formed |
| kinase inhibitor | | | | | | | by cyclin D1-CDK4, cyclin |
| p27 (p27kip1) | | | | | | | A-CDK2 and cyclin E-CDK2. |
| | | | | | | | Overexpression causes G1 |
| | | | | | | | arrest in cell cycle. p27kip1 |
| | | | | | | | has a region of sequence |
| | | | | | | | similarity to the p21 cyclin- |
| | | | | | | | Cdk inhibitor (p21Cip1/WAF1) |
| | | | | | | | and may mediate TGF-b-induced |
| | | | | | | | G1 arrest. |
| | | | | | | | Medline 8033212, 8033213, |
| | | | | | | | 9264403, |
| | | | | | | | Swiss-Prot P46414, |

Job 9846
Comment 1: Downregulation of p27kip1 has been proposed to modulate the adaptive growth of
cardiomyocytes during pressure overload-induced LVH. Am J Physiol 273:H1358-67, 1997.
04.01.01 Fatty Acid Synthesis

| | | | | | | | |
|---|---|---|---|---|---|---|---|
| Cytosolic Malic | m30596 | g1i0- | — | — | +1.8 | +2 | Malic enzyme (also called |
| enzyme: Rat | | 192.8 | | | | | NADP+—linked malate enzyme) |
| cytosolic malic | | | | | | | catalyzes the oxidative |
| enzyme | | | | | | | decarboxylation of Malate to |
| | | | | | | | Pyruvate and produces NADPH |
| | | | | | | | from NADP+. NADPH is then |
| | | | | | | | used as the reducing agent |
| | | | | | | | and a source of hydrogens for |
| | | | | | | | chain elongation in fatty |
| | | | | | | | acid synthesis. This reac- |
| | | | | | | | tion occurs in the cytosol |
| | | | | | | | and is involved in the |
| | | | | | | | process of bringing Acetyl |
| | | | | | | | CoA from the mitochondria |
| | | | | | | | to the cytosol for fatty |
| | | | | | | | acid synthesis. In this |
| | | | | | | | process, Oxaloacetate and |
| | | | | | | | Acetyl CoA are condensed to |
| | | | | | | | form Citrate which is trans- |
| | | | | | | | ported into the cytosol. Once |
| | | | | | | | in the cytosol, Citrate is |
| | | | | | | | cleaved back into Acetyl CoA |
| | | | | | | | and Oxaloacetate. The Oxalo- |
| | | | | | | | acetate is then converted |
| | | | | | | | into Malate and then Pyruvate |
| | | | | | | | (by Malic enzyme) to be |
| | | | | | | | transported back into the |
| | | | | | | | mitochondria and recycled. |

04.01.02.01 Mitochondrial Beta Oxidation

| | | | | | | | |
|---|---|---|---|---|---|---|---|
| HAD: Rat L-3- | af095449 | s0c0- | — | — | −1.7 | −1.8 | Third step in mitochondrial |
| hydroxyacyl-CoA | | 214 | | | | | beta oxidation. Catalyzes the |
| dehydrogenase | | | | | | | oxidation of the hydroxyl |
| precursor | | | | | | | group at C-3 into a keto |
| | | | | | | | group and generates NADPH. |
| | | | | | | | This enzyme is specific for |
| | | | | | | | the L-isomer of the hydroxy- |
| | | | | | | | acyl substrate. |
| ECH1: Rat | d16478 | m1n0- | — | — | −2.3 | −7 | Second step in mitochondrial |
| mitochondrial | | 385.5 | | | | | beta oxidation. Catalyzes the |
| long-chain enoyl- | | | | | | | stereospecific hydration |
| CoA hydratase | | | | | | | between C-2 and C-3 of Enoyl |
| | | | | | | | CoA. |
| | | | | | | | Swiss-Prot Q62651, |
| ECHB: Rat | d16479 | d0g0- | — | — | −3.1 | −2.3 | Final step in mitochondrial |
| mitochondrial | | 101 | | | | | beta oxidation. Uses CoA-SH |
| long-chain 3- | | g0m0- | | | | | to cleave the 3-ketoacyl-CoA |
| ketoacyl-CoA | | 105.3 | | | | | freeing up acetyl-CoA to go |
| thiolase | | | | | | | into the Krebs cycle and |
| | | | | | | | adding a new CoA group onto |
| | | | | | | | the now-exposed 3-keto group |
| | | | | | | | to create an acyl-CoA n-2 |
| | | | | | | | shorter than the previous |
| | | | | | | | acyl-CoA and this restarts |
| | | | | | | | the beta oxidation cycle. |
| | | | | | | | Swiss-Prot Q60587, |
| MCAD: Rat | j02791 | m1n0- | — | — | −2.1 | −2.2 | Short-chain, medium-chain, |
| medium chain | | 202.4 | | | | | and long-chain acyl-CoA |
| acyl-CoA | | | | | | | dehydrogenases catalyze the |
| dehydrogenase | | | | | | | initial reaction in the beta- |
| | | | | | | | oxidation of fatty acids. |
| | | | | | | | Medium chain acyl-CoA |
| | | | | | | | dehydrogenase covers the |
| | | | | | | | initial dehydrogenation of |
| | | | | | | | C4-C12 straight chain acyl- |
| | | | | | | | CoA's in mitochondrial beta |
| | | | | | | | oxidation. (EC1.3.99.3) |
| | | | | | | | OMIM 201450, |
| | | | | | | | Swiss-Prot P11310, |

04.03.01 Glycolysis/Gluconeogenesis

| | | | | | | | |
|---|---|---|---|---|---|---|---|
| Beta beta | gber_aa851223 | m1n0- | +1.9 | +1.9 | — | — | 521 bp EST with 98% identity |
| enolase-seq var: | | 181.2 | | | | | to rattus norvegicus beta |
| EST encoding | | | | | | | beta enolase (y00979). |
| beta beta | | | | | | | |
| enolase-sequence | | | | | | | |
| variant, cloned | | | | | | | |
| from rat placenta. | | | | | | | |

Job 9838
Comment 1: AA851223 is a 521 bp EST with 98% identity to beta beta enolase (y00979). Contains a point
mutation difference from y00979 that creates a RE site and fragment m1n0-181.2, not seen in y00979.

| | | | | | | | |
|---|---|---|---|---|---|---|---|
| Beta beta | y00979 | m0r0- | +2.1 | +1.5 | — | — | Rat muscle-specific beta- |
| enolase: Rat | | 251.6 | | | | | enolase (beta beta enolase). |
| muscle-specific | | | | | | | Enzyme involved in the last |
| beta-enolase | | | | | | | stage of the glycolytic |
| (beta beta | | | | | | | pathway, catalyzes the de- |
| enolase). [C] | | | | | | | hydration of 2-phosphogly- |
| | | | | | | | cerate to phosphoenolpyruvate |
| | | | | | | | (one step prior to the |
| | | | | | | | formation of pyruvate and |
| | | | | | | | generation of the second ATP |
| | | | | | | | molecule). |

04.04.03 ATP/Proton Motive Force Interconversion

| | | | | | | | |
|---|---|---|---|---|---|---|---|
| UCP2: Rat UCP2 | ab010743 | g0m0- | −1.4 | −1.7 | −1.6 | −1.8 | UCP2 (uncoupling protein 2) |
| | | 120.7 | | | | | is 59% homologous to UPC1. |
| | | | | | | | UPCs are transmembrane |
| | | | | | | | proteins of the inner mito- |
| | | | | | | | chondrial membrane that can |
| | | | | | | | uncouple ATP production from |
| | | | | | | | mitochondrial respiration, |
| | | | | | | | allowing energy to be |
| | | | | | | | released as heat and de- |
| | | | | | | | creasing energy metabolism |
| | | | | | | | efficiency. Unlike UCP1 and |
| | | | | | | | UCP3, UCP2 has a wide tissue |
| | | | | | | | distribution including |
| | | | | | | | heart, kidney, lung, |
| | | | | | | | placenta, lymphocytes and |
| | | | | | | | white fat. |

Job 9838
Comment 1: Down-regulated in both exercise and RVhtn hypertrophy jobs.

| | | | | | | | |
|---|---|---|---|---|---|---|---|
| UCP1: Rat | x03894 | l0n0- | — | — | +4 | +3.7 | UCP1 (uncoupling protein 1) |
| nuclear mRNA for | | 181.2 | | | | | is a transmembrane protein |
| mitochondrial | | m0s0- | | | | | found in the mitochondrial |
| uncoupling | | 91.7 | | | | | inner membrane. When |
| protein | | | | | | | activated by cold, UPC1 can |
| (UCP1) (mapping | | | | | | | allow hydrogen ions to pass |
| request made) | | | | | | | through the inner mito- |
| | | | | | | | chondrial membrane, abol- |
| | | | | | | | ishing the hydrogen ion |
| | | | | | | | gradient necessary to drive |
| | | | | | | | ATP synthesis which raises |
| | | | | | | | the body's metabolic rate |
| | | | | | | | and generates heat. The |
| | | | | | | | protein is normally kept in |
| | | | | | | | an inactive state by nucleo- |
| | | | | | | | tides that bind the protein. |
| | | | | | | | UPC1 expression is |
| | | | | | | | restricted to brown adipose |
| | | | | | | | tissue and induced upon |
| | | | | | | | birth, cold acclimation and |
| | | | | | | | overfeeding. |

Job 9846
Comment 1: Poisoning is only a pass partial. Looks like modulation is about 30% of total modulation
for the band. (28% modulation was seen in the poisoning reaction done on the control samples.)
04.05.02 Aspartate Family

| | | | | | | | |
|---|---|---|---|---|---|---|---|
| ASAT: Rat | d00252 | l0m0- | — | — | −1.8 | −1.7 | During amino acid degrada- |
| cytosolic | | 403.3 | | | | | tion, the alpha-amino group |
| aspartate | | | | | | | of many amino acids is |
| aminotransferase | | | | | | | transferred to 2-oxoglutarate |
| (also called | | | | | | | (alpha-ketoglutarate) by an |
| Transaminase A | | | | | | | aminotransferase to form |
| or glutamate | | | | | | | glutamate. Aspartate amino- |
| oxaloacetate | | | | | | | transferase catalyzes the |
| transaminase-1) | | | | | | | transfer of the amino group |
| | | | | | | | of aspartate to 2oxoglutarate |
| | | | | | | | using pyridoxal-phosphate as |
| | | | | | | | a cofactor. L-ASPARTATE + |
| | | | | | | | 2-OXOGLUTARATE = OXALO- |
| | | | | | | | ACETATE + L-GLUTAMATE. In |
| | | | | | | | eukaryotes, there are two |
| | | | | | | | isozymes: a cytoplasmic form |
| | | | | | | | and a mitochondrial form. |
| | | | | | | | Swiss-Prot P13221, |

04.08.03 Catecholamine Metabolism

| | | | | | | | |
|---|---|---|---|---|---|---|---|
| MAO-A: Rat | d00688 | f0i0- | −1.4 | −1.8 | −1.7 | −2 | Monoamine oxidase A (MAO-A) |
| monoamine | | 172.2 | | | | | belongs to the flavin mono- |
| oxidase A | | g1n0- | | | | | amine oxidase family and |
| | | 106.9 | | | | | catalyzes the oxidative |
| | | | | | | | deamination of biogenic and |
| | | | | | | | xenobiotic amines and has |
| | | | | | | | important functions in the |
| | | | | | | | metabolism of neuroactive |
| | | | | | | | and vasoactive amines in |
| | | | | | | | the central nervous system |
| | | | | | | | and peripheral tissues. |
| | | | | | | | MAO-A preferentially |
| | | | | | | | oxidizes biogenic amines |
| | | | | | | | such as 5-hydroxytryptamine |
| | | | | | | | (5-HT), norepinephrine and |
| | | | | | | | epinephrine. It is localized |
| | | | | | | | to the mitochondrial outer |
| | | | | | | | membrane. |
| | | | | | | | Swiss-Prot P21396, |

Job 9846
Comment 1: Catecholamines control myocardial contractibility by interactions with the beta adrenergic
receptors and adenylate cyclase system.
04.11.02.06 Water

| | | | | | | | |
|---|---|---|---|---|---|---|---|
| AQP1:Rat | X67948 | u0g1- | — | — | +1.9 | +1.8 | Aquaporin 1 is a six-trans- |
| channel integral | | 235.5 | | | | | membrane domain protein that |
| membrane protein | | | | | | | is present in many fluid |
| 28. | | | | | | | secreting and absorbing |
| | | | | | | | tissues including kidney, |
| | | | | | | | brain, heart and eye. Forms |
| | | | | | | | a water-specific channel that |
| | | | | | | | provides the plasma membranes |
| | | | | | | | of red cells and kidney |
| | | | | | | | proximal tubules with high |
| | | | | | | | permeability to water, |
| | | | | | | | thereby permitting water to |
| | | | | | | | move in the direction of an |
| | | | | | | | osmotic gradient. |
| | | | | | | | Pharmacologically inhibited |
| | | | | | | | by submillimolar concentra- |
| | | | | | | | tions of Hg2+. |
| | | | | | | | Medline 97445992, Swiss-Prot |
| | | | | | | | P29975, |

Job 34877
Comment 1: Aquaporin1/CHIP28 has also been identified to be expressed in freshly dispersed,
differentiated, cultured rat aortic vascular smooth muscle cells but not highly expressed in other
smooth muscle tissues. PMID: 8393626
Comment 2: There is some data to support the regulation of AQP1 by arginine vasopressin (AVP) and
atrial natriuretic peptide (ANP). PMID:9299519
05.01.01 Components

| | | | | | | | |
|---|---|---|---|---|---|---|---|
| Sim to human | ai103318 | g1g0- | −1.9 | −1.8 | — | — | Rat EST (536 bp) cloned from |
| Sop2-like: Rat | | 117.8 | | | | | normalized rat embryo. |
| EST similar to | | | | | | | Similar to human Sop2-like |
| human Sop2-like | | | | | | | protein (y08999)- 92% over |
| gene cloned from | | | | | | | 198 bp and 98% over 62aa. |
| normalized | | | | | | | ORF with similarity to y08999 |
| embryo | | | | | | | is encoded by nucleotides 1 |
| | | | | | | | to 186 in frame −1 of EST |
| | | | | | | | ai103318. Sop2 (suppressor of |
| | | | | | | | profilin-2) was first cloned |
| | | | | | | | in S. pombe, is a member of |
| | | | | | | | the WD-repeat containing |
| | | | | | | | protein family and is |
| | | | | | | | presumed to have a role in |
| | | | | | | | cortical actin-requiring |
| | | | | | | | processes (shown to interact |
| | | | | | | | with actin-related protein |
| | | | | | | | (Arp3), profilin and actin.) |
| | | | | | | | EST could not be extended. |
| | | | | | | | Medline 8978670, |

05.01.01.03 Structural Arm: Actins & Short Filaments

| | | | | | | | |
|---|---|---|---|---|---|---|---|
| ACTA: Rat | v01218 | g0c0- | +1.5 | +1.5 | +2.1 | +2.1 | Actins are highly conserved |
| skeletal muscle | | 294.7 | | | | | proteins that are involved in |
| actin [C] | | | | | | | various types of cell |
| | | | | | | | motility and are ubiquitously |
| | | | | | | | expressed in all eukaryotic |
| | | | | | | | cells. In vertebrates, 3 main |
| | | | | | | | groups of actin isoforms |
| | | | | | | | (alpha, beta and gamma) have |
| | | | | | | | been identified. Alpha actins |
| | | | | | | | are found in muscle tissues |
| | | | | | | | and are a major constituent |
| | | | | | | | of the contractile apparatus |
| | | | | | | | while the beta and gamma |
| | | | | | | | actins are primarily |
| | | | | | | | components of the cyto- |
| | | | | | | | skeleton and mediate internal |
| | | | | | | | cell motility. Alpha actins |
| | | | | | | | bind to myosin in the muscle |
| | | | | | | | myofibrils and facilitate |
| | | | | | | | release of ADP from myosin |
| | | | | | | | following ATP hydrolysis |
| | | | | | | | during muscle contraction. |
| | | | | | | | Swiss-Prot p02568, |

Job 9846
Comment 1: Skeletal alpha actin may account for up to half of the actin mRNA in an adult heart with
cardiac alpha actin MrNA making up the rest (Mol Cell Bid 3:1985-95, 1983). However, increased
expression of skeletal alpha actin has long been known to be a marker of cardiac hypertrophy (J Clin
Invest 80:1194-9, 1987; PNAS 88:2132-6, 1991; Circ Res 72:857-64, 1993.
Comment 2: Cardiac alpha actin is reported as a partial fragment for rattus norvegicus (x00306). It
contains 1 predicted band, k0n0-116, that was present in exercise, RVhtn and control animals. The
complete cardiac alpha actin is reported for rattus rattus (X80130), and it is 98% identical to the
partial fragment reported for rattus norvegicus. This rattus rattus fragment contains the same
predicted band, k0n0-116, plus 3 additional bands: i0l0-217, l0e1-367 and m0v0-37. These additional
bands may be slightly decreased in the RVhtn animal (−1.5), but do not seem to be changed in the
exercise animals.
05.01.01.04 Structural Arm: Heavy Filaments

| | | | | | | | |
|---|---|---|---|---|---|---|---|
| MYH6: Rat alpha | x15938 | l0w0- | — | — | −3.6 | −3 | Muscle myosin is a hexameric |
| cardiac myosin | | 131.1 | | | | | protein that consists of 2 |
| heavy chain | | | | | | | heavy chain subunits (MHC), |
| | | | | | | | 2 light chain subunits (MLC), |
| | | | | | | | and 2 regulatory light chain |
| | | | | | | | subunits (MLC-2). Muscle |
| | | | | | | | myosin is found in the thick |
| | | | | | | | filaments of the myofibrils. |
| | | | | | | | ATP hydrolysis drives muscle |
| | | | | | | | contraction which is produced |
| | | | | | | | by the sliding of actin |
| | | | | | | | filaments against myosin |
| | | | | | | | filaments. Myosin is an |
| | | | | | | | actin-activated ATPase. There |
| | | | | | | | are two cardiac myosin heavy |
| | | | | | | | chain isoforms, alpha and |
| | | | | | | | beta, encoded by two separate |
| | | | | | | | genes that are 4kb apart in |
| | | | | | | | the rat genome. The cardiac |
| | | | | | | | alpha isoform is a ‘fast’ |
| | | | | | | | ATPase, while the beta |
| | | | | | | | isoform is a ‘slow’ ATPase. |
| | | | | | | | Swiss-Prot P02563, |

Job 9846
Comment 1: Decreased mechanical performance has been reported to be due to decreased myosin
ATPase activity in various models of hemodynamic load (alpha-MHC has the highest ATPase activity and
beta-MHC has the lowest). A switch from alpha- to beta-MHC isozyme in response to cardiac overload
has previously been reported. In this study, we are seeing a decrease in expression of alpha-MHC, but
no change in beta-MHC expression.
05.01.01.05 Binding Proteins

| | | | | | | | |
|---|---|---|---|---|---|---|---|
| CFL1: Rat cofilin | x62908 | l0k0- | — | — | −11.2 | −9.5 | Cofilin is an actin modulated |
| | | 326 | | | | | protein that is widely |
| | | | | | | | distributed throughout muscle |
| | | | | | | | and non-muscle cells. Cofilin |
| | | | | | | | controls actin polymerization |
| | | | | | | | and depolymerization in a pH- |
| | | | | | | | sensitive manner and has the |
| | | | | | | | ability to bind g-and f-actin |
| | | | | | | | in a 1:1 ratio of cofilin to |
| | | | | | | | actin. Cofilin is the major |
| | | | | | | | component of intranuclear and |
| | | | | | | | cytoplasmic actin rods. Two |
| | | | | | | | cofilin isoforms exist in |
| | | | | | | | mammals, muscle type and |
| | | | | | | | non-muscle type. Muscle type |
| | | | | | | | was detected predominantly in |
| | | | | | | | heart, skeletal muscle, C2 |
| | | | | | | | myotubes and testis by |
| | | | | | | | Northern blot while non- |
| | | | | | | | muscle type was seen in a |
| | | | | | | | variety of non-muscle |
| | | | | | | | tissues. There is only a |
| | | | | | | | single cofilin sequence |
| | | | | | | | available for rat. |
| | | | | | | | Medline 8195165, |
| | | | | | | | Swiss-Prot P45592, |

Job 9846
Comment 1: Although non-muscle and muscle isoforms have been reported for mouse and human, only a
single cofilin sequence is publicly available for rat. The rat sequence is more similar to the general
mouse cofilin sequence (non-muscle) than to the muscle specific sequence. There are no reports in the
literature of modulation in cofilin expression in association with cardiac hypertrophy.
05.01.03.03 Contractile CA + 2 Regulators

| | | | | | | | |
|---|---|---|---|---|---|---|---|
| CASQ2: Rat | af001334 | r0w0- | −18.3 | −11.6 | −18.5 | −14.2 | Calsequestrin is a high- |
| cardiac | | 434.5 | | | | | capacity, moderate affinity |
| calsequestrin | | r0w0- | | | | | Ca(2+)-binding protein |
| | | 434.5 | | | | | localized in the terminal |
| | | | | | | | cisternae luminal spaces of |
| | | | | | | | the sarcoplasmic reticulum of |
| | | | | | | | skeletal and cardiac muscle |
| | | | | | | | cells. Calsequestrin |
| | | | | | | | functions as a Ca(2+) |
| | | | | | | | storage protein in the lumen |
| | | | | | | | of the sarcoplasmic |
| | | | | | | | reticulum. The release of |
| | | | | | | | calcium bound to calseques- |
| | | | | | | | trin through a calcium |
| | | | | | | | release channel triggers |
| | | | | | | | muscle contraction. Two |
| | | | | | | | isoforms of calsequestrin |
| | | | | | | | have been identified, cardiac |
| | | | | | | | and skeletal, which both have |
| | | | | | | | high acidic amino acid |
| | | | | | | | composition. The two isoforms |
| | | | | | | | are very similar except for |
| | | | | | | | the carboxy-terminus. The |
| | | | | | | | cardiac isoform is expressed |
| | | | | | | | in heart and slow skeletal |
| | | | | | | | muscle while the skeletal |
| | | | | | | | muscle isoform is expressed |
| | | | | | | | in fast and slow skeletal |
| | | | | | | | muscle. Medline 7816057, |
| | | | | | | | Swiss-Prot P51868, |

Job 9838
Comment 1: Calsequestrin sequence poisoned two bands, r0w0-434.5 (found only in control samples)
and r0w0-430.0 (found in hypertrophy samples and control samples). Sequence is found in the 3′ UTR of
calsequestrin, and this band is the only band for this gene that can be identified using GeneCalling.
There have been reports of a reduced ability of the sarcoplasmic reticulum to accumulate Ca2+\in
heart failure, but no expression changes in calsequestrin have previously been identified in
hypertrophy models.
05.10 Others

| | | | | | | | |
|---|---|---|---|---|---|---|---|
| Sim to mouse | gber_aa799471 | u0f0- | +2.8 | +2.5 | +1.4 | +1.5 | Telethonin is a sarcomeric |
| telethonin: Rat | | 69.2 | | | | | protein expressed in heart |
| EST (656 bp) | | u0f0- | | | | | and skeletal muscle (very |
| similar to mouse | | 69.3 | | | | | abundant transcript). Shown |
| telethonin | | | | | | | to interact with titin/ |
| (AJ223854) (87% | | | | | | | connectin in a confirmation |
| identical, 3″end of | | | | | | | dependent manner in Y2H |
| CDS) | | | | | | | analyses and may be phos- |
| | | | | | | | phorylated by titin/ |
| | | | | | | | connectin, suggesting a role |
| | | | | | | | in myofibrillogenesis. |
| | | | | | | | Specific to heart and muscle |
| | | | | | | | tissue. Previously cloned in |
| | | | | | | | human and mouse but sequence |
| | | | | | | | is not yet reported for rat. |
| | | | | | | | Swiss-Prot 015273, |

Job 9838
Comment 1: 87% identical to mouse telethonin (aj223854) gene, 58% identical to mouse telethonin
protein (translated).
07.02.03 Ion Pumps

| | | | | | | | |
|---|---|---|---|---|---|---|---|
| SERCA-2: Rat | af043106 | d0v0- | — | — | −2.7 | −1.8 | The SERCA Ca(2+)-ATPases |
| sarco/endoplasmic | | 133.9 | | | | | are intracellular pumps |
| reticulum Ca2+- | | | | | | | located in the sarcoplasmic |
| ATPase (SERCA- | | | | | | | or endoplasmic reticula of |
| 2) | | | | | | | muscle cells (integral |
| | | | | | | | membrane protein). SERCA2 |
| | | | | | | | belongs to the large family |
| | | | | | | | of P-type cation pumps that |
| | | | | | | | couple ATP hydrolysis with |
| | | | | | | | cation transport across |
| | | | | | | | membranes. SERCA pumps |
| | | | | | | | specifically maintain low |
| | | | | | | | cytosolic Ca(2+) concen- |
| | | | | | | | trations by actively trans- |
| | | | | | | | porting Ca(2+) from the |
| | | | | | | | cytosol into the sarco/ |
| | | | | | | | endoplasmic reticulum lumen. |
| | | | | | | | ATP2A2 encodes 2 alterna- |
| | | | | | | | tively spliced transcripts, |
| | | | | | | | encoding isoforms SERCA2a and |
| | | | | | | | SERCA2b, respectively. |
| | | | | | | | SERCA2a and SERCA2b differ |
| | | | | | | | in their carboxy termini and |
| | | | | | | | have distinct tissue- |
| | | | | | | | expression patterns. SERCA2a |
| | | | | | | | is located primarily in heart |
| | | | | | | | and slow-twitch skeletal |
| | | | | | | | muscle, whereas SERCA2b is |
| | | | | | | | present in smooth muscle and |
| | | | | | | | nonmuscle tissues. |
| | | | | | | | OMIM 108740, |
| | | | | | | | Swiss-Prot P11508, |

Job 9846
Comment 1: Decreases in SERCA2a have been reported in hypertrophied cardiac tissue due to aortic
stenosis. Circulation 98(22):2477-86, 1998.
09 Unknown Function

| | | | | | | | |
|---|---|---|---|---|---|---|---|
| Sim to mouse | cgrnl0y0157.2_9838- | l0y0- | +2.1 | +2.1 | — | — | Novel rat gene fragment (157 |
| Nocturnin: 92% | 73 | 157.2 | | | | | bp) with similarity to mus |
| Sim. to Mus | | | | | | | musculus probable nocturnin |
| musculus probable | | | | | | | gene (u70139), 92% identity |
| nocturnin (U70139) | | | | | | | at the nucleotide level and |
| [N] | | | | | | | 100% identity at the amino |
| | | | | | | | acid level. This novel rat |
| | | | | | | | gene fragment also has 77% |
| | | | | | | | identity at the nucleotide |
| | | | | | | | level and 80% identity at the |
| | | | | | | | amino acid level to Xenopus |
| | | | | | | | laevis nocturnin (u74761), |
| | | | | | | | identified as a retina mRNA |
| | | | | | | | that is expressed in peak |
| | | | | | | | abundance at night. Nocturnin |
| | | | | | | | has strong sequence |
| | | | | | | | similarity to the C-terminal |
| | | | | | | | domain of the yeast tran- |
| | | | | | | | scription factor CCR4 as well |
| | | | | | | | as a leucine zipper-like |
| | | | | | | | dimerization motif and is |
| | | | | | | | presumed to be a component of |
| | | | | | | | the circadian clock or down- |
| | | | | | | | stream effector of clock |
| | | | | | | | function. |
| | | | | | | | Medline 8962150, 9038221, |

Job 9838
Comment 1: Sequence could not be further extended

| | | | | | | | |
|---|---|---|---|---|---|---|---|
| Cgrnw0c0282_9838- | cgrnw0c0282_9838- | w0c0- | +2.6 | +2.5 | — | — | Novel 281 bp fragment that |
| 301: Partially | 301 | 282 | | | | | extends EST Al407719 (630 bp) |
| novel, extends rat | | | | | | | by 23 bp to form a 653 bp |
| EST Al407719 [N] | | | | | | | contig. No significant |
| | | | | | | | homologies to genes of known |
| | | | | | | | function at the DNA or |
| | | | | | | | protein level. ORFs are found |
| | | | | | | | in 5 reading frames with the |
| | | | | | | | longest in frame + |
| | | | | | | | 3 (101aa). |

Job 9838
Comment 1: Novel 281 bp fragment that extends EST Al407719 by 23 bp to form a 653 bp contig:

| | |
|---|---|
| GTCTAATGTCAGGGCGAAATCAAGCCCACGGCAAAGAATTATGAGACATCCCCAGGCACCAGG | (SEQ ID NO.1) |
| CTCACACTCCCAGGGCAGGACCAAAGACTGATGCCTAGAGCGGGTAAGGGGTGTCGTGGGTGTCCCTGAG | |
| AAGCTCAGTC | |
| CAGAGGGCCTTTGTCTAAGAGACTCTGAGAAAGGGATGGGTGGCAGGAAGCTTGGGGAATAAGGGTATTAA | |
| GAAGAGAAT | |
| AAATTAAAGGGGGGGCTTGAGGGACAAGGGGCCTGTGCTGTCCTTCAAACAGCTGGGAGCAGACCAGGGG | |
| TGGGAAAGAG | |
| GGTGGCGGGAAGAGCTTGATACACTATCTTAAGAAACACCGTTTACCCACTTCCCTCTTAACCACTGCAGTG | |
| CACAACGA | |
| GCCAGGGCACAGGGCAGGAGCCCACATGCCCCAGTGGCTTTCAACATGGCACGGGTATAAAGGGAAGCAG | |
| CTGAGGGATA | |
| TCTCAGGAAGGGGAAGTTATCCCCTGGTCCCCAAATGCTATAAGGCACAATTCTTGGAGGCAACTAGATTCC | |
| ATCCAAAA | |
| TATTAAAGGAAAAAAAAAAACAACTTCAAAACAGAAAACTTTAAATCCCAGGTCTACTGTGACTTCGCTTGGGC | |
| CTGGTC AACACTCACCTAGCATCACAGGGGGCTAGC | |

| | | | | | | | |
|---|---|---|---|---|---|---|---|
| Cgrny0k0141.7_9838- | cgrny0k0141.7_9838- | y0k0- | +1.9 | +1.5 | −1.7 | −1.7 | 141 bp gene fragment from rat |
| 126: Partially | 126 | 141.6 | | | | | that overlaps with rat EST |
| novel, extends rat | | y0k0- | | | | | H34237(310 bp) and extends |
| EST gber_h34237 | | 141.7 | | | | | the EST by 57 bp creating a |
| [N] | | | | | | | 367 bp contig. Contig encodes |
| | | | | | | | a 122aa ORF in frame-1 that |
| | | | | | | | has 84% identity at the |
| | | | | | | | nucleotide level and |
| | | | | | | | approximately 70% identity |
| | | | | | | | at the amino acid level to |
| | | | | | | | human protein KlAA0025- |
| | | | | | | | 392aa(D14695), a predicted |
| | | | | | | | transmembrane protein with |
| | | | | | | | unknown function. |

Job 9838
Comment 1: 141 bp isolated fragment overlaps with rat EST H34237, creating a 367 bp contig:

| | |
|---|---|
| CGGCTGCATTCTGAATTGGCCGGCTGGTTTTCTGCCGGAAACTGGTTGTGTAATAGGGNCGGGAGCCGGTG | (SEQ ID NO.2) |
| TAGAGACCA | |
| CAGGTATTTCTTGTGCACTTGGTGTAGGGCCAAAAGCTCCTGAAGCAGCAGTGGCAGCCAAGTATTGCATGT | |
| AGTACTNT | |
| CTTGCATAGATCTGCTGGAACCAGGAGAGCTGCAGGCCACCCGTAGGTGGTGTAGCCAAAGAANCCGGGC | |
| CCGAGGCCTT | |
| GGAAAGTCTGCTGGACGGCTTCAGGCCTAGAGACGTTCTCCCATCCAGAGGGAGGAAGGTTCCGAAGGACT | |
| TCCCTTTCC CGTAAGCCATCGCTTGAGGAATCCCCAGGATACTGTGCCTGACTAGT | |

| | | | | | | | |
|---|---|---|---|---|---|---|---|
| Cgrny0n0162.9_9838- | cgrny0n0.162.9₁₃ 9839- | y0n0- | +2.6 | −2.5 | — | — | Novel 163 bp fragment that |
| 115: Partially | 115 | 162.9 | | | | | extends EST AA819672 (485 bp) |
| novel, extends rat | | | | | | | 53 bp to form a 538 bp |
| EST AA819672 [N] | | | | | | | contig. Contig contains a |
| | | | | | | | 53aa ORF in frame − 1 starting |
| | | | | | | | at nuc 1. No significant |
| | | | | | | | homologies to genes of known |
| | | | | | | | function at the DNA or |
| | | | | | | | protein level. Does contain |
| | | | | | | | strong homology to a 10aa |
| | | | | | | | motif found in glutamine |
| | | | | | | | amidotransferase class-II |
| | | | | | | | proteins (esp. rat amino- |
| | | | | | | | phosphoribosyltransferase- |
| | | | | | | | D10853). |

Job 9838
Comment 1: Novel 163 bp fragment that extends EST AA819672 53 bp to form a 538 bp contig:

| | |
|---|---|
| TTTTTTTTTTTTTTTTTAGACCCATATTAGGTTTATTTAATAACAGAGCACTCGCTTCTTTAAATAAAATATCTCA | (SEQ ID NO.3) |
| AAGT | |
| TCTAGCTTTGCCTCAAACACAATGTTGCACCCAAACAGAAAAGCACAAATCAAACCAACAGAAAGATAGTTTT | |
| TTTTAAA | |
| AAATTATCTCCTTAGGCCTCTGTCTTTAACTTCCCCTTGTTCCTATTTCTATGAGAGAGACCGTAACGCACAGG | |
| CTGAGG | |
| AGACACACTGCCAACAAGGCTAATGTGCACCAGACCGAAGAGGGACAGCTCGGCTTTGGCCAGCCCTCTTC | |
| CTGCAGGAT | |
| ACCAATCCTATGTTTGCGTCAATCCTGACCTGCTCAGATGAAGCGGCACTCAGGCACTAGTCAGCCGTTGAC | |
| CATACAAG | |
| AACAGAGAACACTGGAGTAGACAGAGCTTTCTCCAGGAATGCTGACAGGCGTCCCTCCCTTTTGAGAAGTCC | |
| TTTGCTTT CCTGACCCCTGTGCTTCAGGCACCCTGGCAAGGCCAACCAACTTCCTTCAGCTGTACA | |

| | | | | | | | |
|---|---|---|---|---|---|---|---|
| SM-20: Rat SM-20 | u06713 | f0k0-66 | — | — | −4.1 | −2.9 | Identified by differential |
| | | | | | | | screening of a rat aortic |
| | | | | | | | smooth muscle cell cDNA |
| | | | | | | | library to be induced |
| | | | | | | | following treatment with |
| | | | | | | | growth agonists (serum, PDGF, |
| | | | | | | | angiotensin II). Expressed at |
| | | | | | | | high levels in muscle cells |
| | | | | | | | (smooth, skeletal and cardiac |
| | | | | | | | as well as the brain - not |
| | | | | | | | found in fibroblasts.) The |
| | | | | | | | protein localizes to |
| | | | | | | | filaments in the cytoplasm of |
| | | | | | | | smooth muscle cells. In the |
| | | | | | | | brain, see increased |
| | | | | | | | expression in sympathetic |
| | | | | | | | neurons deprived of nerve |
| | | | | | | | growth factor (NGF)-proposed |
| | | | | | | | role in regulation of |
| | | | | | | | neuronal cell death. Text |
| | | | | | | | article, |
| 09.04 Novel | | | | | | | |
| Cgrng0y0369_9846- | cgrng0y03969_9846- | g0y0- | — | — | −4 | −2 | Novel rat gene fragment (369 |
| 106: Novel - | 106 | 369 | | | | | bp). No similarity to any |
| Sim. to mouse EST | | | | | | | known genes at the nucleotide |
| AA286474 [N] | | | | | | | or amino acid levels. ORFs |
| | | | | | | | all 6 reading frames. |
| | | | | | | | Sequence could not be |
| | | | | | | | extended. |

Job 9846
Comment 1: Novel 369 bp fragment:

| | |
|---|---|
| ggatccagtttgagacagtagctgttatgagatcactgcttctcctcatcttgctcctgagcacaggctgggagaagaca | (SEQ ID NO. 4) |
| tggggtgcactggggtatccttgtagaatagcagcctttattccttcctcaccttacctgtctctggaagggcagcaatt | |
| gcatgggacaattaacatctggacttagcctctgatcatagctcagaaccttctggaatagcgggttagaggacagaatt | |
| ttcagagcagggctgtttggtcaagaggtggacttaagccttccttgggcttcacttggccaactgaggcactcacaatg | |
| tcccctcacgtctctgtcaatccaggttgacccccatctttggactagt | |

| | | | | | | | |
|---|---|---|---|---|---|---|---|
| Cgrng1k0329.8_9838- | cgrng1k0329.8_9839- | g1k0- | −6.4 | −4.8 | −7.7 | −5.2 | Novel rat gene fragment (327 |
| 225: | 225 | 329.8 | | | | | bp). No similarity to any |
| Novel [N] | | g1k0- | | | | | known genes at the nucleotide |
| | | 329.8 | | | | | or amino acid levels. ORFs |
| | | | | | | | all 6 reading frames. |
| | | | | | | | Sequence could not be |
| | | | | | | | extended. |

Job 9838
Comment 1: Novel 327 bp fragment:

| | |
|---|---|
| tctagaacctgtgaagtccagggagaagggagcacagtggccgtgggtgccactggcctcccagggaagcccttagatat | (SEQ ID NO. 5) |
| caccagtgtgcacagcagagcagcacacgtgtgtacgtgtgtgtatgtgtgtgtgcatgtgtgtgtatgtatgcctgggt | |
| ccatgccggtgactgggcattggagggtctagggagggcaggactacagggactcctgcttggactgagccttcctacag | |
| cctaggtagcctgtgtggctccagagccaggtagtcgtggtctctgtattagctggtcaggggaggcagtgaggggtatg tgggccc | |

References

Abu-Shakra, S. R., Cole, A. J. and Drachman, D. B. Nerve stimulation and denervation induce differential patterns of immediate early gene mRNA expression in skeletal muscle. Brain Res Mel Brain Res 18, 216-20 (1993).

Bandoh, S., Tsukada, T., Maruyama, K., Ohkura, N. and Yamaguchi, K. Differential expression of NGFI-B and RNR-1 genes in various tissues and developing brain of the rat: comparative study by quantitative reverse transcription-polymerase chain reaction. J Neuroendocrinol 9, 3-8 (1997).

Crawford, P. A., Sadovsky, Y., Woodson, K., Lee, S. L. and Milbrandt, J. Adrenocortical function and regulation of the steroid 21-hydroxylase gene NGFI-B-deficient mice. Mol Cell Biol 15, 4331-16 (1995).

Feo, S., Antona, V., Barbieri, G., Passantino, R., Cali, L., and Giallongo, A. Transcription of the human beta enolase gene (ENO-3) is regulated by an intronic muscle-specific enhancer that binds myocyte-specific enhancer factor 2 proteins and ubiquitous G-rich-box binding factors. Mol Cell Biol 15, 5991-6002 (1995).

Fonjallaz, P., Ossipow, V., Wanner, G. and Schibler, U. The two PAR leucine zipper proteins, TEF and DBP, display similar circadian and tissue-specific expression, but have different target promoter preferences. EMBO J 15, 351-62 (1996).

Hayashi, K., Ohkura, N., Miki, K., Osada, S. and Tomino, Y. Early induction of the NGFI-B/Nur77 family genes in nephritis induced by anti-glomerular basement membrane antibody. Mol Cell Endocrinol 123, 205-9 (1996).

Keller, A., Rouzeau, J.-D., Farhadian, F., Wisnewsky, C., Marotte, F., Lamande, N., Samuel, J.-L., Schwartz, K., Lazar, M. and Lucas, M. Differential expression of alpha- and beta-enolase genes during rat heart development and hypertrophy. Am J Physiol 269, H1843-51 (1995).

Lim, R. W., Zhu, C. Y. and Stringer, B. Differential regulation of primary response gene expression in skeletal muscle cells through multiple signal transduction pathways. Biochim Biosphys Acta 1266, 91-100 (1995).

Liu, Z. G., Smith, S. W., McLaughlin, K. A., Schwartz, L. M. and Osborne, B. A. Apoptotic signals delivered through the T-cell receptor of a T-cell hybrid require the immediate-early gene nur77. Nature 367, 281-4 (1994).

Maruyama, K., Tsukada, T., Ohkura, N., Bandoh, S., Hosono, T. and Yamaguchi, K. The NGFI-B subfamily of the nuclear receptor superfamily. Int J Oncol 12, 1237-43 (1998).

Milbrandt, J. Nerve growth factor induces a gene homologous to the glucocorticoid receptor gene. Neuron 1, 183-8 (1988).

Morris, M. E., Viswanathan, N., Kuhlman, S., Davis, F. C. and Weitz, C. J. A screen for genes induced in the suprachiasmatic nucleus by light. Science 279, 1544-7 (1998).

Mueller, C. R., Maire, P. and Schibler, U. DBP, a liver-enriched transcriptional activator, is expressed late in ontogeny and its tissue specificity is determined posttranscriptionally. Cell 61, 279-291 (1990).

Ohshima, Y., Mitsui, H., Takayama, Y., Kushiya, E., Sakimura, K. & Takahashi, Y. cDNA Cloning and Nucleotide Sequence of Rat Muscle-Specific Enolase (Beta Beta Enolase). FEBS Lett 242, 425-30 (1989).

Park, E. A., Song, S., Olive, M. and Roesler, W. J. CCAT-enhancer-binding protein alpha (C/EBP alpha) is required for the thyroid hormone but not the retinoic acid induction of phosphoenolpyruvate carboxykinase (PEPCK) gene transcription. Biochem J 15, 343-9 (1997).

Roesler, W. J., McFie, P. J. and Dauvin, C. The liver-enriched transcription factor D-site-binding protein activates the promoter of the phosphoenolpyruvate carboxykinase gene in hepatoma cells. J Biol Chem 267, 21235-43 (1992).

Rusak, B., McNaughton, L., Robertson, H. A. and Hunt, S. P. Circadian variation in photic regulation of immediate-early gene mRNAs in rat suprachiasmatic nucleus cells. Brain Res Mol Brain Res 14, 124-30 (1992).

Sakimura, K., Kushiya, E., Ohshima-lchimura, Y., Mitsui, H. & Takahashi, Y. Structure and expression of rat muscle-specific enolase gene. FEBS Lett 277, 78-82 (1990).

Ueda, Y., Bandoh, S., Fujita, J., Sato, M., Yamaji, Y. and Takahara, J. Expression of nerve growth factor-induced clone B subfamily and pro-opiomelanocortin gene in lung cancer cell lines. Am J Respir Cell Mol Biol 20, 1319-(1999).

Van den Hoff, M. J., Vermeulen, J. L., De Boer, P. A., Lamers, W. H. and Moorman, A. F. Developmental changes in the expression of the liver-enriched transcription factors LF-B1, C/EBP, DBP and LAP/LIP in relation to the expression of albumin, alpha-fetoprotein, carbamoylphosphate synthase and lactase mRNA. Histochem J 26, 20-31 (1994).

Watson, W. A. and Milbrandt, J. The NGFI-B gene, a transcriptionally inducible member of the steroid receptor gene superfamily: genomic structure and expression in rat brain after seizure induction. Mol Cell Biol 9, 4213-9 (1989).

Williams, G. T. and Lau, L. F. Activation of the inducible orphan receptor gene nur77 by serum growth factors: dissociation of immediate-early and delayed-early responses. Mol Cell Biol 13, 6124-36 (1993).

Wilson, T. E., Fahrner, T. J., Johnston, M and Milbrandt, J. Identification of the DNA binding site for NGFI-B by genetic selection in yeast. Science 252, 1296-1300 (1991).

Woronicz, J. D., Calnan, B., Ngo, V. and Winoto, A. Requirement for the orphan steroid receptor Nur77 in apoptosis of T-cell hybridomas. Nature 367, 277-81 (1994).

That which is claimed is:

1. A method of screening a compound for effects on cardiac muscle, comprising:
(a) administering a test compound to a test animal;
(b) obtaining a sample of heart tissue from said animal; and
(c) determining the level of expression of a gene selected from the group consisting of uncoupling protein 1 (UCP1), cytosolic malic enzyme, and atrial natriuretic factor (ANP);
where increased expression of said gene in the test animal, compared to expression that would occur in a control animal, indicates said compound induces cardiac hypertrophy.

2. A method according to claim 1 where said test animal is a rodent.

3. A method according to claim 1 where said test animal is selected from mice and rats.

4. A method of screening a compound for effects on cardiac muscle, comprising:
(a) administering a test compound to a test animal;
(b) obtaining a sample of heart tissue from said animal; and
(c) determining the level of expression of a gene selected from the group consisting of cofilin, cyclin-dependent kinase inhibitor p27 (p27kip1), smooth muscle 20 (SM-20), alpha cardiac myosin heavy chain (MYH6), mitochondrial long-chain 3-ketoacyl-CoA thiolase, medium chain acyl-CoA dehydrogenase, sarco/endoplasmic reticulum Ca2+ATPase (SERCA-2), and cyclin G;
where decreased expression of said gene in the test animal, compared to expression that would occur in a control animal, indicates said compound induces cardiac hypertrophy.

5. A method according to claim 1 where said test animal is a rodent.

6. A method according to claim 1 where said test animal is selected from mice and rats.

7. A method of diagnosing cardiac hypertrophy in an animal, comprising:
(a) obtaining a sample of heart tissue from said animal; and
(b) determining at least one of the following:
(i) whether the level of expression of a gene selected from the group consisting of cofilin, cyclin-dependent kinase inhibitor p27 (p27kip1), smooth muscle 20 (SM-20), alpha cardiac myosin heavy chain (MYH6), mitochondrial long-chain 3-ketoacyl-CoA thiolase, medium chain acyl-CoA dehydrogenase, sarco/endoplasmic reticulum Ca2+ATPase (SERCA-2), and cyclin G is decreased compared to normal levels of expression;
(ii) whether the level of expression of a gene selected from the group consisting of uncoupling protein 1 (UCP1), cytosolic malic enzyme, and atrial natriuretic factor (ANP) is increased compared to normal levels of expression;
(iii) whether the level of expression of a gene selected from the group consisting of nerve growth factor-induced-B (NGFI-B), D-binding protein, Thyrotroph Embryonic Factor (TEF), beta beta enolase, nocturnin, and deubiquitinating enzyme (UBP45) is increased compared to normal levels of expression;
where increased expression of a gene of (I) or decreased expression of a gene of (ii) indicate that the hypertrophy is pathologic, and increased expression of a gene of (iii) indicate that the hypertrophy is physiologic.


SEQUENCE LISTING (5 sequences)

SEQ ID NO: 1 - DNA, 653 bp, Rattus rattus
gtctaatgtcagggcgaaatcaagcccacggcaaagaattatgagacatccccaggcaccaggctcacactcccagggcaggaccaaagactgatgcctagagcgggtaaggggtgtcgtgggtgtccctgagaagctcagtccagagggcctttgtctaagagactctgagaaagggatgggtggcaggaagcttggggaataagggtattaagaagagaataaattaaagggggggcttgagggacaaggggcctgtgctgtccttcaaacagctgggagcagaccaggggtgggaaagagggtggcgggaagagcttgatacactatcttaagaaacaccgtttacccacttccctcttaaccactgcagtgcacaacgagccagggcacagggcaggagcccacatgccccagtggctttcaacatggcacgggtataaagggaagcagctgagggatatctcaggaaggggaagttatcccctggtccccaaatgctataaggcacaattcttggaggcaactagattccatccaaaatattaaaggaaaaaaaaaaacaacttcaaaacagaaaactttaaatcccaggtctactgtgacttcgcttgggcctggtcaacactcacctagcatcacagggggctagc

SEQ ID NO: 2 - DNA, 367 bp, Rattus rattus
  misc_feature 59, 159, 224: n = A,T,C or G
cggctgcattctgaattggccggctggttttctgccggaaactggttgtgtaatagggncgggagccggtgtagagaccacaggtatttcttgtgcacttggtgtagggccaaaagctcctgaagcagcagtggcagccaagtattgcatgtagtactntcttgcatagatctgctggaaccaggagagctgcaggccacccgtaggtggtgtagccaaagaanccgggcccgaggccttggaaagtctgctggacggcttcaggcctagagacgttctcccatccagagggaggaaggttccgaaggacttccctttcccgtaagccatcgcttgaggaatccccaggatactgtgcctgactagt

SEQ ID NO: 3 - DNA, 538 bp, Rattus rattus
tttttttttttttttttagacccatattaggtttatttaataacagagcactcgcttctttaaataaaatatctcaaagttctagctttgcctcaaacacaatgttgcacccaaacagaaaagcacaaatcaaaccaacagaaagatagttttttttaaaaaattatctccttaggcctctgtctttaacttccccttgttcctatttctatgagagagaccgtaacgcacaggctgaggagacacactgccaacaaggctaatgtgcaccagaccgaagagggacagctcggctttggccagccctcttcctgcaggataccaatcctatgtttgcgtcaatcctgacctgctcagatgaagcggcactcaggcactagtcagccgttgaccatacaagaacagagaacactggagtagacagagctttctccaggaatgctgacaggcgtccctcccttttgagaagtcctttgctttcctgacccctgtgcttcaggcaccctggcaaggccaaccaacttccttcagctgtaca

SEQ ID NO: 4 - DNA, 369 bp, Rattus rattus
ggatccagtttgagacagtagctgttatgagatcactgcttctcctcatcttgctcctgagcacaggctgggagaagacatggggtgcactggggtatccttgtagaatagcagcctttattccttcctcaccttacctgtctctggaagggcagcaattgcatgggacaattaacatctggacttagcctctgatcatagctcagaaccttctggaatagcgggttagaggacagaattttcagagcagggctgtttggtcaagaggtggacttaagccttccttgggcttcacttggccaactgaggcactcacaatgtcccctcacgtctctgtcaatccaggttgacccccatctttggactagt

SEQ ID NO: 5 - DNA, 327 bp, Rattus rattus
tctagaacctgtgaagtccagggagaagggagcacagtggccgtgggtgccactggcctcccagggaagcccttagatatcaccagtgtgcacagcagagcagcacacgtgtgtacgtgtgtgtatgtgtgtgtgcatgtgtgtgtatgtatgcctgggtccatgccggtgactgggcattggagggtctagggagggcaggactacagggactcctgcttggactgagccttcctacagcctaggtagcctgtgtggctccagagccaggtagtcgtggtctctgtattagctggtcaggggaggcagtgaggggtatgtgggccc